(19) United States
(12) Patent Application Publication
(10) Pub. No.: US 2021/0180045 A1
(43) Pub. Date: Jun. 17, 2021

(54) SCALABLE TAGGING OF ENDOGENOUS GENES BY HOMOLOGY-INDEPENDENT INTRON TARGETING

(71) Applicant: THE CHILDREN'S HOSPITAL OF PHILADELPHIA, PHILADELPHIA, PA (US)

(72) Inventors: Ophir H. SHALEM, Philadelphia, PA (US); Yevgeniy V. SEREBRENIK, Philadelphia, PA (US); Stephanie E. SANSBURY, Philadelphia, PA (US)

(73) Assignee: THE CHILDREN'S HOSPITAL OF PHILADELPHIA, PHILADELPHIA, PA (US)

(21) Appl. No.: 17/272,005

(22) PCT Filed: Sep. 3, 2019
(86) PCT No.: PCT/US2019/049267

§ 371 Date: Feb. 26, 2021

Related U.S. Application Data
(60) Provisional application No. 62/725,714, filed on Aug. 31, 2018.

(51) Int. Cl. C12N 15/10; C12N 9/22

(52) U.S. Cl. CPC C12N 15/1065; C12N 9/22

(57) ABSTRACT

Provided herein are nucleic acid compositions and methods of their use for integrating reporter proteins, such as split fluorophore protein fragments, into intronic genomic regions. The integrated sequence is flanked by a splice acceptor site and a splice donor site such that the reporter protein sequence is incorporated into the mature mRNA expressed from the target gene.

### REFERENCE TO RELATED APPLICATIONS

The present application claims the priority benefit of U.S. provisional application No. 62/725,714, filed Aug. 31, 2018, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Aug. 6, 2019, is named CHOPP0021WO_ST25.txt and is 22 kilobytes in size.

### BACKGROUND

### 1. Field

The present invention relates generally to the field of molecular biology. More particularly, it concerns compositions and methods for tagging target endogenous proteins with reporters by inserting synthetic exons into an intronic sequence of the target protein.

### 2. Description of Related Art

Generating knock-in cell lines in which genes are endogenously fused to fluorescence or epitope tags is a powerful, widely used and essential approach for studying proteins within their natural regulatory context. The advent of CRISPR tools for modifying the genome (Doudna & Charpentier, 2014; Hsu et al., 2014; Ran et al., 2013) has made this easier and even more accessible, yet scalability is still very limited. The need for a gene-specific Homology Directed Repair (HDR) template requires costly synthesis or labor-intensive molecular cloning, and since precise targeting must be achieved in frame with the coding sequence, it necessitates careful design of reagents and screening of clonal cell lines to avoid disruptive editing at the non-tagged allele. The development of split fluorescent proteins has simplified the generation of fluorescent fusions, since only a minimal tag is required for genomic knock-in (Cabantous et al. 2005; Kamiyama et al. 2016; Leonetti et al. 2016; Feng et al. 2017). Nevertheless, these endogenous tagging methods still require individual HDR donors. Several approaches to develop generic exon-tagging methods have been demonstrated (Lackner et al. 2015; Schmid-Burgk et al. 2016), but because these require precise tagging at the coding sequence, they are limited in design flexibility and are prone to disruptive mutations at the nontagged allele as well as to indels within the tagged allele that can lead to frameshifts. Derivative strategies have been developed to increase the efficiency of homology-independent repair-dependent tagging methods but at the cost of no longer utilizing a generic donor (Suzuki et al. 2016).

An alternative approach for generating endogenous fusions is by random integration of synthetic exons delivered by transposons or retroviral particles (Trinh le & Fraser, 2013). This approach, known as “protein trapping” or “CD-tagging” (Jarvik et al., 1996), is not restricted to small donors and has been used in both model organisms (Buszczak et al., 2007; Clyne et al., 2003; Trinh le et al., 2011) and mammalian cells (Cohen et al., 2008; Sigal et al., 2007). While protein trapping is inexpensive and scalable, the random nature of tag integration precludes its use for the generation of curated libraries of fusion cell lines. As such, new methods are needed for generating endogenous gene fusions.

### SUMMARY

As such, provided herein are methods of using a combination of protein trapping and gene targeting to tag genes. This approach, which targets introns, is efficient, easy to implement, and does not limit the size of the donor. Furthermore, in contrast to generic exon tagging, generic intron tagging allows for flexible donor design owing to the splice acceptor and donor sites: Any incorporated vector sequence external to those sites has no effect on the coding sequence of the tagged protein. This property not only enables protein tagging with precisely defined tags, but also allows for the addition of genomic elements, such as those encoding a resistance gene, that do not disrupt the target gene coding sequence. These methods, for example, can be used to introduce antibiotic selection markers in a nondisruptive way to obtain high proportions of positively tagged cells. Generic intron tagging also tolerates mutations in the nontagged allele (because those are intronic and typically nondisruptive) as well as indels that flank the inserted donor as a result of editing that could lead to frameshifts in an exonic setting. Because the donor is generic, the generation of additional fusion cell lines merely requires the cloning of additional intron-targeting sgRNAs. Furthermore, introns provide a wide range of protospacer options to choose from, allowing for the selection of sgRNAs with few off-target effects. The efficiency and flexibility of this system is useful for large-scale tagging experiments, as well as for quickly screening many sites for protein tagging.

In one embodiment, nucleic acid compositions are provided, said nucleic acids comprising, from 5′ to 3′, a first sgRNA binding site, a splice acceptor site, a sequence encoding a reporter protein, a splice donor site, and a second sgRNA recognition/binding site. In some aspects, the first and second sgRNA binding sites comprise the same nucleotide sequence. In some aspects, the reporter protein is a fluorescent protein. In certain aspects, the fluorescent protein is a split fluorescent protein fragment. In some aspects, the nucleic acids further comprise an antibiotic resistance gene (e.g., a blasticidin gene) positioned between the splice donor site and the second sgRNA binding site or between the first sgRNA binding site and the splice acceptor site.

In one embodiment, compositions are provided, said compositions comprising an endonuclease-encoding nucleic acid sequence, the donor plasmid of any one of the present embodiments, and a donor plasmid-specific gRNA-encoding sequence. In some aspects, the endonuclease is a Cas endonuclease. In some aspects, the Cas endonuclease is a Cas9 endonuclease. In some aspects, the compositions further comprise a site-specific guide RNA (gRNA)-encoding nucleic acid sequence. In some aspects, the site-specific and/or donor plasmid-specific guide RNA is a single gRNA. In some aspects, the site-specific and/or donor plasmid-specific guide RNA is a CRISPR-RNA (crRNA). In some aspects, the site-specific and/or donor plasmid-specific guide RNA comprises a fusion of a crRNA and a trans-activating CRISPR RNA (tracrRNA). In some aspects, the guide RNA comprises a crRNA and a tracrRNA. In some aspects, the endonuclease and the donor plasmid-specific gRNA are encoded on a single nucleic acid molecule. In some aspects, the endonuclease, the donor plasmid-specific gRNA, and the donor plasmid are encoded on a single nucleic acid molecule. In some aspects, each of the endonuclease-encoding nucleic acid sequence, the donor plasmid, the site-specific guide RNA (gRNA)-encoding nucleic acid sequence, and the donor plasmid-specific gRNA-encoding sequence are present on separate nucleic acid molecules.

In one embodiment, methods are provided for integrating an exogenous DNA sequence into an intronic genomic sequence of a target gene in a cell, the method comprising delivering a composition comprising delivering to the cell a composition of any one of the present embodiments. In some aspects, the portion of the donor plasmid comprising the splice acceptor site, the sequence encoding a reporter protein, and the splice donor site is integrated into the intronic genomic sequence of a target gene. In some aspects, the reporter protein is expressed with the target gene. In some aspects, the portion of the donor plasmid comprising the splice acceptor site, the sequence encoding a reporter protein, the splice donor site, and the antibiotic resistance gene is integrated into the intronic genomic sequence of a target gene. In some aspects, the methods further comprise detecting the expression of the antibiotic resistance gene. In some aspects, the methods further comprise detecting the expression of the reporter protein. In some aspects, the methods comprise integrating the exogenous DNA sequence into an intronic genomic sequence of a second target gene in a second cell. In some aspects, the methods are further defined as a high-throughput method of tagging target genes, wherein the method comprises integrating the exogenous DNA sequence into an intronic genomic sequence in two or more cells, wherein the intronic genomic sequence is unique for each of the two or more cells.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-E: Homology-independent intron tagging enables efficient and easy generation of endogenous fusions. (FIG. 1A) Illustration of the tagging approach. Double-strand breaks are generated in the intron and donor resulting in the addition of a synthetic intron and fusion of the tag to the coding sequence. (FIG. 1B) Using a small donor composed of the mNG2₁₁ epitope flanked by splice acceptor and donor sites results in efficient tagging observed by flow cytometry (upper panels) and by confocal microscopy (lower panels). (FIG. 1C) All transfection mix components are required for tagging. The table indicates which component was removed, and bar plots represent the relative percentage of fluorescence-positive cells compared to the full mix. (FIG. 1D) Tagging using a full-length mClover3 fluorophore as a donor. Note that a difference in localization is observed for ACTB between the small and large tag. (FIG. 1E) Tagging of CANX and CBX1 in HeLa cells, H9 human embryonic stem cells (hESC), and HAP1 cells. All images are maximum projections of Z-stacks, and scale bars correspond to 10 μm.

FIGS. 2A-D: Successful tagging is mostly determined by the choice of intron. (FIG. 2A) Tagging with mNG2₁₁ across introns in ACTB and CANX. Bar plots represent the percent of fluorescence-positive cells for each sgRNA position. (FIG. 2B) Expression mean and standard error for positive cells in each location. Sample sizes are proportional to the bar plots in FIG. 2A. (FIG. 2C) Gel image showing the amplification of donor to genomic DNA junctions, as illustrated in the right-hand diagrams. In the diagrams, black arrows represent primer sites for amplification and red arrows represent primer sites for sequencing in FIG. 2D. (FIG. 2D) Sanger sequencing of donor to genomic DNA junctions shows de-dephasing at the donor and genomic DNA junction, which indicates indels at the integration site.

FIGS. 3A-E: A modified donor allows for easy selection of tagged cells. (FIG. 3A) Schematic of donor constructs without and with a blasticidin resistance (BSD) gene. (FIG. 3B) Enrichment of fluorescence-positive HEK293 mNG2₁₋₁₀ cells tagged with mNG2₁₁-BSD(−/+) at CANX intron 14 and CBX1 intron 3 after blasticidin treatment. Data represent mean±SEM (n=3). (FIG. 3C) Dot plots of total HEK293 cell populations tagged with mNG2₁₁ or with mNG2₁₁-BSD(−/+) and selected for 12 d. Plots are shaded by density. (FIG. 3D) Confocal microscopy of total cell populations as in FIG. 3C. Images are maximum projections of Z-stacks, and scale bars correspond to 10 μm. (FIG. 3E) Western blot of clonal HAP1 lysates tagged with mClover3 only or mClover3-BSD(−/+) at CANX intron 14, target 1. The values below the anti-CANX blot indicate total levels of the major CANX band (tagged and untagged) relative to levels in wild-type (w.t.) cells.

FIG. 4: Additional intronic tagging locations for ACTB and CANX. Microscopy images of HEK293 cells stably expressing mNG2₁₋₁₀ tagged with mNG211 targeting the indicated genomic sites. Images are maximum projections of z-stacks and scale bars correspond to 10 μm.

FIGS. 5A-C: Clonal HAP1 cells tagged with mClover3-only or mClover3-BSD(−/+) at CANX intron 14, sgRNA target 1. (FIG. 5A) PCR analysis of the targeted CANX locus. Wild-type locus is 1921 bp; tagging with mClover3-only or -BSD(−/+) adds multiples of 881 or 1734 bp, respectively (ladder is NEB 1kb N3232). (FIG. 5B) Uncropped Western blot of CANX from FIG. 3E. Molecular weight (MW) of ladder is indicated. (FIG. 5C) Mean fluorescence intensities of mClover3-tagged HAP1 clones measured by flow cytometry.

FIGS. 6A-B: DNA sequences of the mNG2₁₁ (FIG. 6A; SEQ ID NO: 1) and mClover3 (FIG. 6B; SEQ ID NO: 2) donor regions in the pMC-mNG2₁₁ and pMC-mClover3 plasmids. In FIG. 6A (SEQ ID NO: 1), nucleotides 4-23 and 203-222 are the donor plasmid protospacer sequence; nucleotides 52-96 and 145-189 are the linker regions; nucleotides 97-144 are the sequence of mNG2₁₁. In FIG. 6B (SEQ ID NO: 2), nucleotides 4-23 and 869-888 are the donor plasmid protospacer sequence; nucleotides 52-96 and 811-855 are the linker regions; nucleotides 97-810 are the sequence of mClover 3. Splice sites are directly adjacent to the sequences incorporated into the coding sequence. Blue brackets (“[_]”) indicate locations were additional nucleotides may be added to change the frame of the tag.

FIGS. 7A-D: DNA sequences of the mNG2₁₁-BSD(−) (FIG. 7A; SEQ ID NO: 3), mNG2₁₁-BSD(+) (FIG. 7B; SEQ ID NO: 4), mClover3-BSD(−) (FIG. 7C; SEQ ID NO: 5), and mClover3-BSD(+) (FIG. 7D; SEQ ID NO: 6) donor regions. In FIG. 7A (SEQ ID NO: 3), nucleotides 4-23 and 1056-1075 are the donor plasmid protospacer sequence; nucleotides 52-96 and 145-189 are the linker regions; nucleotides 97-144 are the sequence of mNG2₁₁; nucleotides 202-1054 are the blasticidin resistance gene (BSD), EF1a promoter, and SV40 poly(A) sequence. In FIG. 7B (SEQ ID NO: 4), nucleotides 4-23 and 1056-1075 are the donor plasmid protospacer sequence; nucleotides 52-96 and 145-189 are the linker regions; nucleotides 97-144 are the sequence of mNG2₁₁; nucleotides 202-1054 are the blasticidin resistance gene (BSD), EF1a promoter, and SV40 poly(A) sequence. In FIG. 7C (SEQ ID NO: 5), nucleotides 4-23 and 1722-1741 are the donor plasmid protospacer sequence; nucleotides 52-96 and 811-855 are the linker regions; nucleotides 97-810 are the sequence of mClover3; nucleotides 868-1720 are the blasticidin resistance gene (BSD), EF1a promoter, and SV40 poly(A) sequence. In FIG. 7D (SEQ ID NO: 6), nucleotides 4-23 and 1722-1741 are the donor plasmid protospacer sequence; nucleotides 52-96 and 811-855 are the linker regions; nucleotides 97-810 are the sequence of mClover3; nucleotides 868-1720 are the blasticidin resistance gene (BSD), EF1a promoter, and SV40 poly(A) sequence. Splice sites are directly adjacent to the sequences incorporated into the coding sequence. Blue brackets (“[_]”) indicate locations were additional nucleotides may be added to change the frame of the tag.

### DETAILED DESCRIPTION

Tagging endogenous genes with fluorescence or epitope tags is commonly done by directing a double strand break (using CRISPR or any other programmable nucleases) to the 3′ end of a coding sequencing and using a DNA homology template as a repair donor. While genome editing tools have simplified the generation of knock-in gene fusions, the requirement for gene-specific homology directed repair (HDR) templates still hinders scalability.

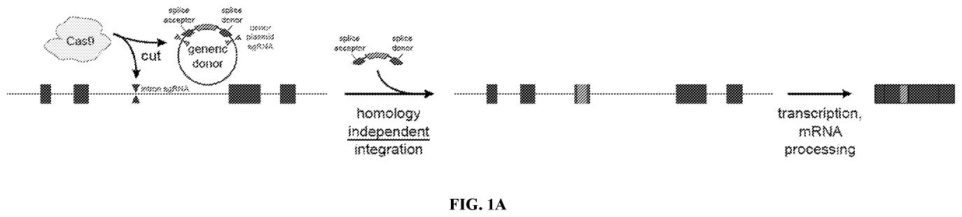

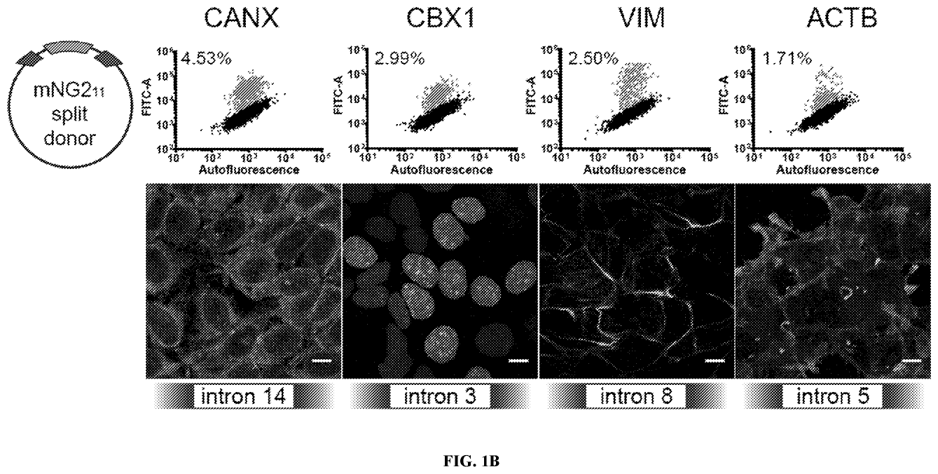

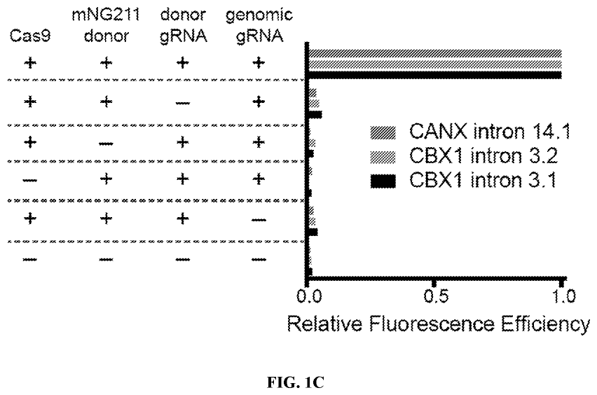

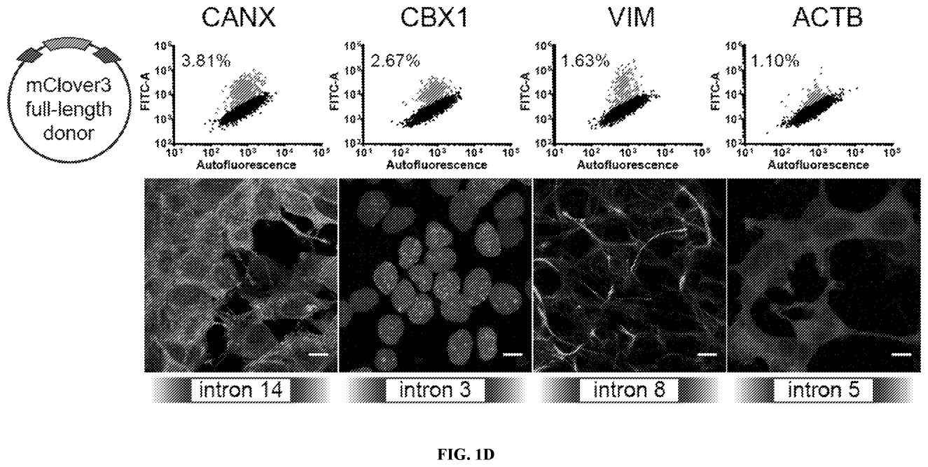

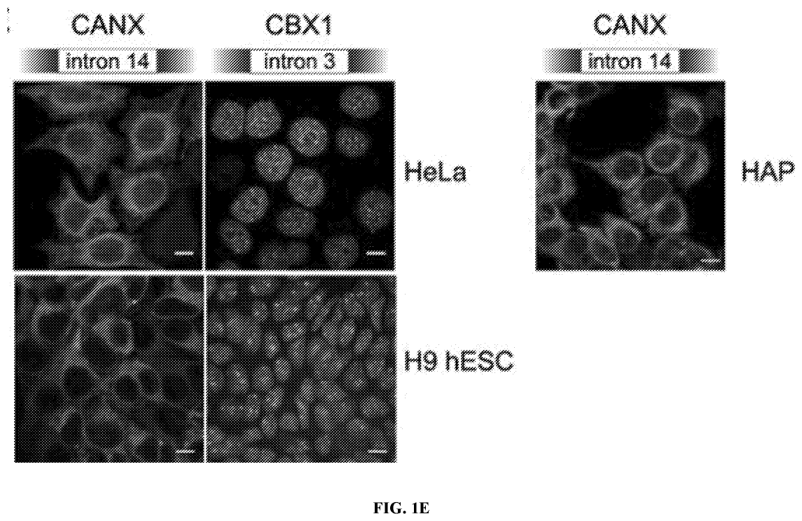

Here, Homology-Independent Targeted Integration (HITI) (Suzuki et al., 2016) is used with a synthetic exon donor containing a fluorescence tag to preform targeted protein trapping at intronic locations (FIG. 1A). In particular, methods are provided for using intron-based protein trapping combined with Homology-Independent Targeted Integration (HITI) to insert a donor flanked by splice acceptor and donor sites. This approach is efficient and easy to implement and does not limit the size of the donor. Furthermore, in contrast to exonic HITI, intronic HITI benefits from increased flexibility of the donor design enabled by the splice acceptor and donor sites: any incorporated vector sequence external to those sites has no effect on the coding sequence of the tagged protein. Intronic HITI also tolerates: (1) mutations in the non-tagged allele, as those are intronic and typically non-disruptive, and (2) indels that flank the inserted donor as a result of HITI-based editing. Most importantly, the donor does not require sequence homology to the insertion site. Because the donor is generic, the generation of additional fusion cell lines only requires the cloning of additional intron-targeting sgRNAs. As such, the present methods provide a means of easy, precise, and efficient gene tagging, which facilitates large-scale interrogation of protein function in the endogenous regulatory context.

### I. ASPECTS OF THE PRESENT INVENTION

Proteins are commonly fused to either fluorescence or epitope tags to study their function, localization, and interactions within living cells. Although exogenous delivery of fused proteins using either plasmid or viral vectors is easy and widely used, results from such experiments are confounded by many factors including overexpression artifacts and the lack of endogenous regulatory context. The advent of easy-to-use genome editing tools has made endogenous tagging much more prevalent but still not common practice. The dependence on HDR limits efficiency and requires costly synthesis of gene-specific HDR templates, which also limits scalability. As such, additional tagging methods are needed, especially those that use generic donors that are better suited for large-scale applications. Here, a tagging strategy is provided that relies on a generic synthetic exon donor. This strategy was used to successfully tag a variety of targets in multiple different cell lines, indicating the general application of this system. Importantly, intronic tagging is largely insensitive to donor size, as tags as small as mNG211 and as large as full-length mClover3 are able to be incorporated. This particular quality of this system stands in contrast to other potentially scalable tagging systems, such as those that are only feasible with small tags and still require donor synthesis (Leonetti et al. 2016).

Methods that use generic donors have been previously demonstrated for N- and C-terminal tagging (Lackner et al. 2015; Schmid-Burgk et al. 2016), yet because tagging is performed directly in the coding sequence, these tools are limited in design flexibility and are prone to disruptive indel mutations both in the tagged and nontagged allele. Compared to the more restricted N- and C-terminal tagging method, intron tagging as described here expands the possible locations for tag integration within the coding region. It is likely that for many proteins, N- or C-terminal tagging would be disruptive, whereas an exposed, nonterminal location would result in a viable fusion. Indeed, internal tagging has borne out various protein- and gene-trap libraries constructed by random intronic integration (Sigal et al. 2006; Buszczak et al. 2007; Bürckstümmer et al. 2013). Successful tagging was largely determined by the relative position of the tag site within the protein-coding region, further emphasizing the importance of tag location within the protein. More information will be needed to better understand how to integrate tags within proteins in the least disruptive way, and the scalability of the intron tagging approach described here will enable systematic tagging experiments to better understand those rules.

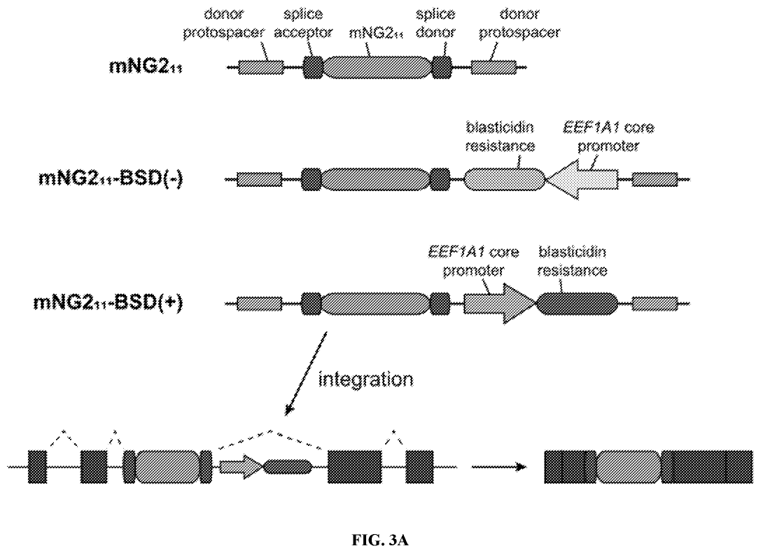

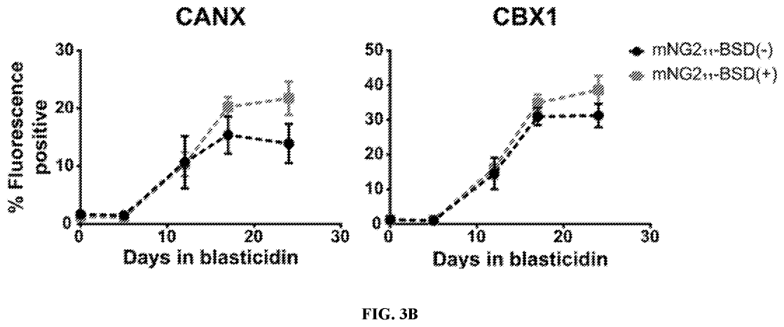

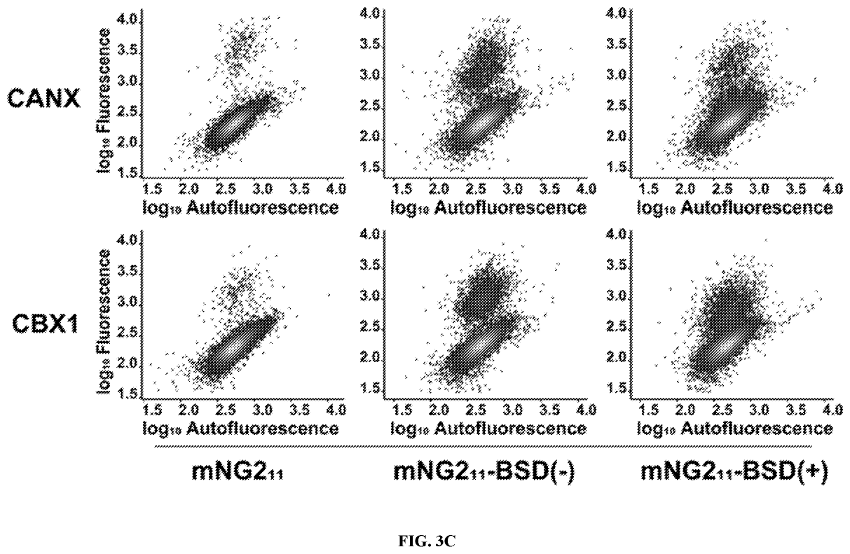

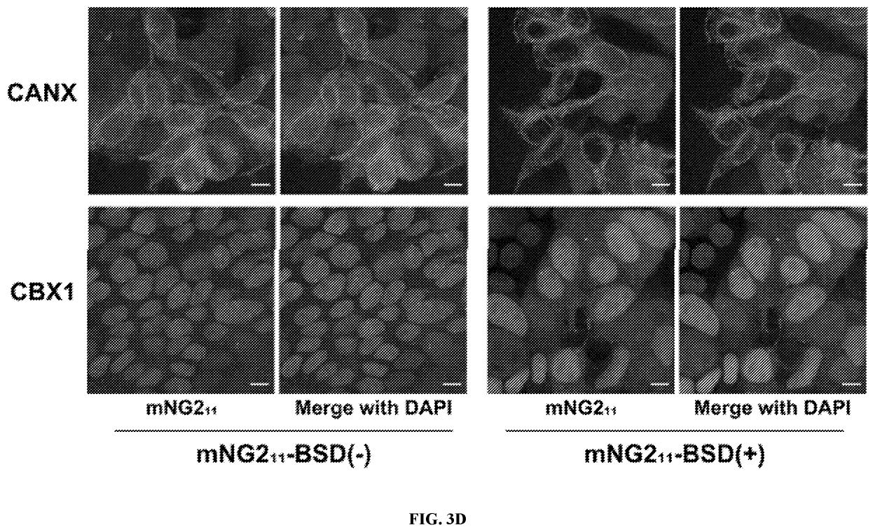

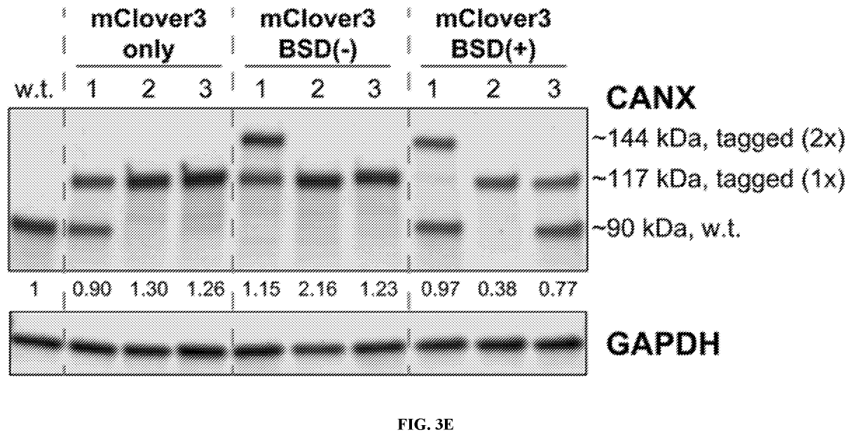

The realization of large-scale tagging experiments depends on the ability to achieve efficient tagging for each gene. Intron tagging increases the number of available protospacer sequences, enabling the selection of the most efficient sgRNAs. To further increase apparent tagging efficiency, the intronic location of the integrated tags was taken advantage of and a blasticidin resistance marker that will not be fused to the mature tagged protein was added (FIG. 3A). This enabled antibiotic selection for tag sites within the protein-coding sequence and not just those at the C-terminus (Schmid-Burgk et al. 2016). This approach can increase the apparent tagging efficiency to as high as 50% (it is limited by the random orientation of tag integration). This will enable more challenging applications such as tagging at low efficiency sites, isolation of cells without sorting, and direct analysis of polyclonal tagged cells, which are not amenable to clonal isolation.

Genomic integration of tags to study protein function at the endogenous context will continue to be vital in cell biology research. As more tools to simplify tagging become available, it will become a common practice to avoid artifacts associated with exogenous overexpression. Generic tagging methods are especially attractive because they enable large-scale tagging at minimal cost. Provided herein is an easy, flexible, scalable and robust method for gene tagging that will help open the door toward the interrogation of proteome dynamics at scale both in arrayed and pooled formats.

### II. EXEMPLARY DETAILED PROTOCOL FOR GENERATING AND ISOLATING FLUORESCENT CELL Clones by Homology-Independent Intron Targeting

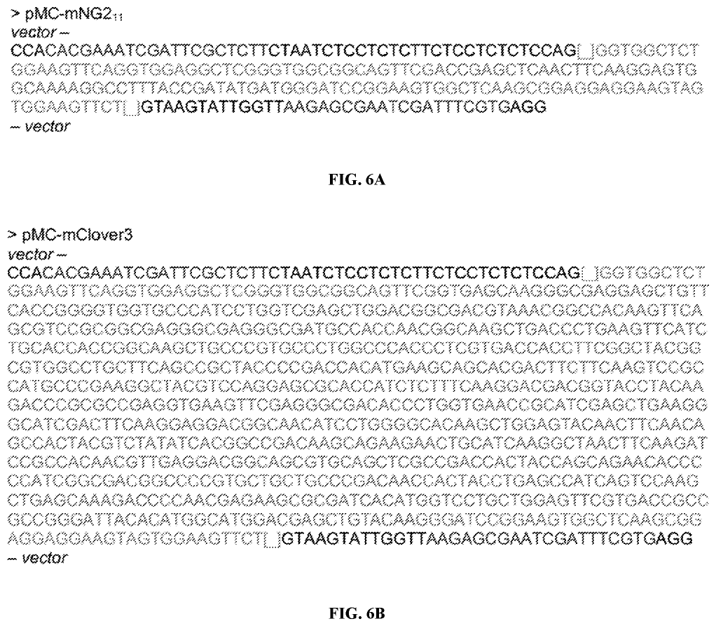

Materials and Reagents:
HEK293 cells (ATCC CRL-1573)
   Can also work with (but not limited to) HeLa cells, H9 hESCs, or HAP1 cells.
Cell culture reagents (Thermo Fischer Scientific): DMEM, trypsin, Opti-MEM
Transfection-grade PEI (Polysciences, cat. #24765)
Plasmid encoding generic fluorescent tag donor
   Any vector can work as long as it contains the sequences listed in FIGS. 6A-B or 7A-D.
   Consider whether the tag will be spliced in-frame with the coding sequence; if not, use a donor with the appropriate frameshift mutations on either end.
Plasmid encoding validated target sgRNA
   The appropriate spacer sequences can be cloned into the lentiGuide-Puro plasmid (Addgene #52963). sgRNA sequences can be chosen using the sgRNA designer provided by the Broad institute (available at portals.broadinstitute.org/gpp/publidanalysis-tools/sgma-design).
Plasmid encoding sgRNA against donor
   See Table 1 for the utilized spacer sequence.
Plasmid encoding Cas9
   Can be used separately, such as on the lentiCas9-Blast plasmid (Addgene #53962), or included on the sgRNA plasmids (e.g. Addgene #52961).

Equipment:
Cell sorter (BD FACSAria Fusion or any other similar cell sorter)
Flow cytometry tubes (12×75 mm)
Cell Straining Kit (70 μm)
Cell culture plates (12-well plate, 10 cm dish, 96-well plate)
Cell culture incubator (37° C., 5% CO₂).

Procedure:
Day 1: Plate cells: 1) Plate HEK293 cells into at least 2 wells of a 12-well plate, such that they will be 60-80% confluent on the following day (160-200×10³ cells/well).
Day 2: Transfect cells” 2) To 100 μl Opti-MEM, add plasmids encoding the generic donor, the donor sgRNA, the target sgRNA, and Cas9 in a 5:1:1:1 molar ratio, respectively. A total amount of 1360 ng of plasmid DNA is mixed with 4.08 μl PEI (1 μg DNA:3 μl PEI ratio). 3) An additional reaction with PEI reagent only is used as the “mock” transfection. 4) Vortex and incubate transfection mixtures for at least 15 minutes. 5) Add each mixture dropwise to a single well of a 12-well plate.
Day 3: Wash and replate cells: 6) Trypsinize cells in 100 μl trypsin, resuspend in 500 μl DMEM, and transfer entire cell suspensions to a 10 cm dish containing 10 ml DMEM.
Day 7-8: Analyze and sort cells: 7) Prepare a 96-well plate filled with 100 μl DMEM per well. Store in cell culture incubator. 8) Trypsinize cells in 1 ml trypsin and resuspend in 2.5 ml DMEM. 9) Pass cell suspension through a cell strainer into a flow cytometry tube (this step is important to prevent clogs in the flow cytometer). Place tube immediately on ice. 10) Initialize cell sorter according to the manufacturer's protocol. Set up gates to differentiate between the “mock”- and truly-transfected cells. 11) Sort single fluorescent cells directly into a 96-well plate. Place immediately in cell culture incubator to grow and expand.

### III. CRISPR/CAS SYSTEMS

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Casl, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Cscl, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

Compositions provided herein comprise targeting constructs comprising at least one targeting sequence. In some embodiments, the targeting construct comprises at least two targeting sequences. Targeting sequences herein are nucleic acid sequences recognized and cleaved by a nuclease disclosed herein in a sequence specific manner. In some embodiments, the targeting sequence is about 9 to about 12 nucleotides in length, from about 12 to about 18 nucleotides in length, from about 18 to about 21 nucleotides in length, from about 21 to about 40 nucleotides in length, from about 40 to about 80 nucleotides in length, or any combination of subranges (e.g., 9-18, 9-21, 9-40, and 9-80 nucleotides). In some embodiments, the targeting sequence comprises a nuclease binding site. In some embodiments the targeting sequence comprises a nick/cleavage site. In some embodiments, the targeting sequence comprises a protospacer adjacent motif (PAM) sequence.

In some embodiments, the target nucleic acid sequence (e.g., protospacer) is 20 nucleotides. In some embodiments, the target nucleic acid is less than 20 nucleotides. In some embodiments, the target nucleic acid is at least 5, 10, 15, 16, 17, 18, 19, 20, 21, 22, 23, 24, 25, 30 or more nucleotides. The target nucleic acid, in some embodiments, is at most 5, 10, 15, 16, 17, 18, 19, 20, 21, 22, 23, 24, 25, 30 or more nucleotides. In some embodiments, the target nucleic acid sequence is 16, 17, 18, 19, 20, 21, 22, or 23 bases immediately 5′ of the first nucleotide of the PAM. In some embodiments, the target nucleic acid sequence is 16, 17, 18, 19, 20, 21, 22, or 23 bases immediately 3′ of the last nucleotide of the PAM. In some embodiments, the target nucleic acid sequence is 20 bases immediately 5′ of the first nucleotide of the PAM. In some embodiments, the target nucleic acid sequence is 20 bases immediately 3′ of the last nucleotide of the PAM. In some embodiments, the target nucleic acid sequence is 5′ or 3′ of the PAM.

A targeting sequence, in some embodiments includes nucleic acid sequences present in a target nucleic acid to which a nucleic acid-targeting segment of a complementary strand nucleic acid binds. For example, targeting sequences, in some embodiments, include sequences to which a complementary strand nucleic acid is designed to have base pairing. A targeting sequence in some embodiments comprises any polynucleotide, which is located, for example, in the nucleus or cytoplasm of a cell or within an organelle of a cell, such as a mitochondrion or chloroplast. Targeting sequences include cleavage sites for nucleases. A targeting sequence, in some embodiments, is adjacent to cleavage sites for nucleases.

The nuclease cleaves the nucleic acid, in some embodiments, at a site within or outside of the nucleic acid sequence present in the target nucleic acid to which the nucleic acid-targeting sequence of the complementary strand binds. The cleavage site, in some embodiments, includes the position of a nucleic acid at which a nuclease produces a single-strand break or a double-strand break. For example, formation of a nuclease complex comprising a complementary strand nucleic acid hybridized to a protease recognition sequence and complexed with a protease results in cleavage of one or both strands in or near (e.g., within 1, 2, 3, 4, 5, 6, 7, 8, 9, 10, 19, 20, 23, 50, or more base pairs from) the nucleic acid sequence present in a target nucleic acid to which a spacer region of a complementary strand nucleic acid binds. The cleavage site, in some embodiments, is on only one strand or on both strands of a nucleic acid. In some embodiments, cleavage sites are at the same position on both strands of the nucleic acid (producing blunt ends) or are at different sites on each strand (producing staggered ends). Staggered ends, in some embodiments, are 5′ or 3′ overhang sticky-ends. Staggered ends, in some embodiments, are produced by sticky-end producing nucleases (e.g., Cpfl). In some embodiments, staggered ends are produced, for example, by using two nucleases, each of which produces a single-strand break at a different cleavage site on each strand, thereby producing a double-strand break. For example, a first nickase creates a single-strand break on the first strand of double-stranded DNA (dsDNA), and a second nickase creates a single-strand break on the second strand of dsDNA such that overhanging sequences are created. In some cases, the nuclease recognition sequence of the nickase on the first strand is separated from the nuclease recognition sequence of the nickase on the second strand by at least 2, 3, 4, 5, 6, 7, 8, 9, 10, 15, 20, 25, 30, 40, 50, 75, 100, 250, 500, or 1000 base pairs.

Site-specific cleavage of a target nucleic acid by a nuclease, in some embodiments, occurs at locations determined by base-pairing complementarity between the complementary strand nucleic acid and the target nucleic acid. Site-specific cleavage of a target nucleic acid by a nuclease protein, in some embodiments, occurs at locations determined by a short motif, called the protospacer adjacent motif (PAM), in the target nucleic acid. For example, the PAM flanks the nuclease recognition sequence at the 3′ end of the recognition sequence. For example, the cleavage site of the nuclease, in some embodiments, is about 1 to about 25, or about 2 to about 5, or about 19 to about 23 base pairs (e.g., 3 base pairs) upstream or downstream of the PAM sequence. In some embodiments, the cleavage site of the nuclease is 3 base pairs upstream of the PAM sequence. In some embodiments, the cleavage site of the nuclease is 19 bases on the (+) strand and 23 base on the (−) strand, producing a 5′ overhang 5 nucleotides (nt) in length. In some cases, the cleavage produces blunt ends. In some cases, the cleavage produces staggered or sticky ends with 5′ overhangs. In some cases, the cleavage produces staggered or sticky ends with 3′ overhangs.

Orthologs of various nuclease proteins utilize different PAM sequences. For example different Cas proteins, in some embodiments, recognize different PAM sequences. For example, in S. pyogenes, the PAM is a sequence in the target nucleic acid that comprises the sequence 5′-XRR-3′, where R is either A or G, where X is any nucleotide and X is immediately 3′ of the target nucleic acid sequence targeted by the spacer sequence. The PAM sequence of S. pyogenes Cas9 (SpyCas9) is 5′-XGG-3′, where X is any DNA nucleotide and is immediately 3′ of the nuclease recognition sequence of the non-complementary strand of the target DNA. The PAM of Cpfl is 5′-TTX-3′, where X is any DNA nucleotide and is immediately 5′ of the nuclease recognition sequence.

### IV. METHODS FOR DELIVERY

Any suitable delivery method is contemplated to be used for delivering the compositions of the disclosure. The individual components of the HITI system (e.g., nuclease and/or the exogenous DNA sequence), in some embodiments, are delivered simultaneously or temporally separated. The choice of method of genetic modification is dependent on the type of cell being transformed and/or the circumstances under which the transformation is taking place (e.g., in vitro, ex vivo, or in vivo).

In some embodiments, a method as disclosed herein involves contacting a target DNA or introducing into a cell (or a population of cells) one or more nucleic acids comprising nucleotide sequences encoding a complementary strand nucleic acid (e.g., gRNA), a site-directed modifying polypeptide (e.g., Cas protein), and/or a exogenous DNA sequence. Suitable nucleic acids comprising nucleotide sequences encoding a complementary strand nucleic acid and/or a site-directed modifying polypeptide include expression vectors, where an expression vector comprising a nucleotide sequence encoding a complementary strand nucleic acid and/or a site-directed modifying polypeptide is a recombinant expression vector.

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and nanoparticle-mediated nucleic acid delivery.

In some aspects, the present disclosure provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a nuclease protein in combination with, and optionally complexed with, a complementary strand sequence is delivered to a cell. Conventional viral and non-viral based gene transfer methods are contemplated to be used to introduce nucleic acids in mammalian cells or target tissues. Such methods are used to administer nucleic acids encoding components of a HITI system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Methods of non-viral delivery of nucleic acids can include lipofection, nucleofection, microinjection, electroporation, biolistics, virosomes, liposomes, immunoliposomes, polycation or lipid:nucleic acid conjugates, naked DNA, artificial virions, and agent-enhanced uptake of DNA.

A host cell is alternatively transiently or non-transiently transfected with one or more vectors described herein. In some embodiments, a cell is transfected as it naturally occurs in a subject. In some embodiments, a cell is taken or derived from a subject and transfected. In some embodiments, a cell is derived from cells taken from a subject, such as a cell line. In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence.

In some embodiments, a nucleotide sequence encoding a complementary strand nucleic acid and/or a site-directed modifying polypeptide is operably linked to a control element, e.g., a transcriptional control element, such as a promoter. The transcriptional control element is functional, in some embodiments, in either a eukaryotic cell, e.g., a mammalian cell, or a prokaryotic cell (e.g., bacterial or archaeal cell). In some embodiments, a nucleotide sequence encoding a complementary strand nucleic acid and/or a site-directed modifying polypeptide is operably linked to multiple control elements that allow expression of the nucleotide sequence encoding a complementary strand nucleic acid and/or a site-directed modifying polypeptide in prokaryotic and/or eukaryotic cells. [0089] Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, etc.).

In some embodiments, a complementary strand nucleic acid and/or a site-directed modifying polypeptide is provided as RNA. In such cases, the complementary strand nucleic acid and/or the RNA encoding the site-directed modifying polypeptide is produced by direct chemical synthesis or may be transcribed in vitro from a DNA encoding the complementary strand nucleic acid. The complementary strand nucleic acid and/or the RNA encoding the site-directed modifying polypeptide are synthesized in vitro using an RNA polymerase enzyme (e.g., T7 polymerase, T3 polymerase, SP6 polymerase, etc.). Once synthesized, the RNA directly contacts a target DNA or is introduced into a cell using any suitable technique for introducing nucleic acids into cells (e.g., microinjection, electroporation, transfection, etc).

### V. KITS AND DIAGNOSTICS

In various aspects of the invention, a kit is envisioned containing the necessary components to insert a reporter encoding sequence in an intron of one or more target gene. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein and will follow substantially the same procedures as described herein or are known to those of ordinary skill. The instruction information may be in a computer readable media containing machine-readable instructions that, when executed using a computer, cause the display of a real or virtual procedure of transfecting or electroporating a CRISPR system in cells in order to insert a reporter encoding sequence in an intron of one or more target gene.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

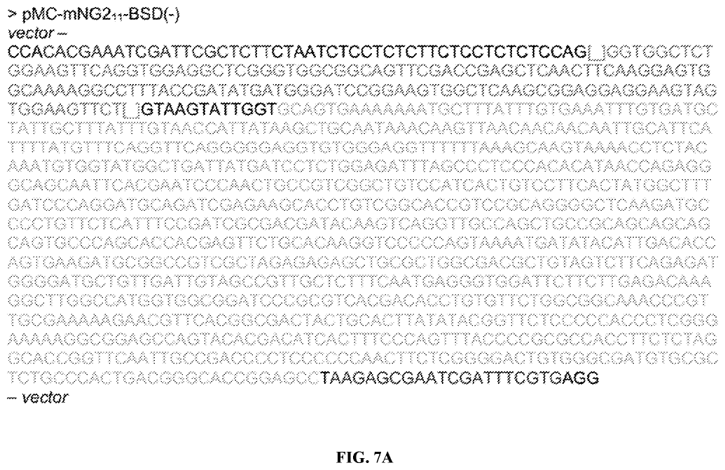

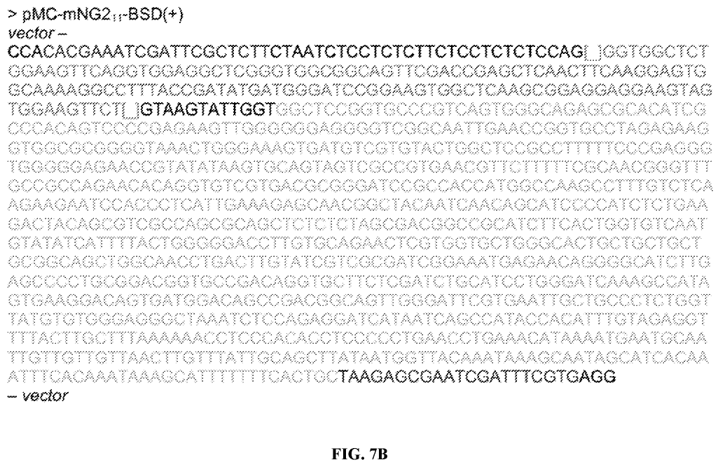

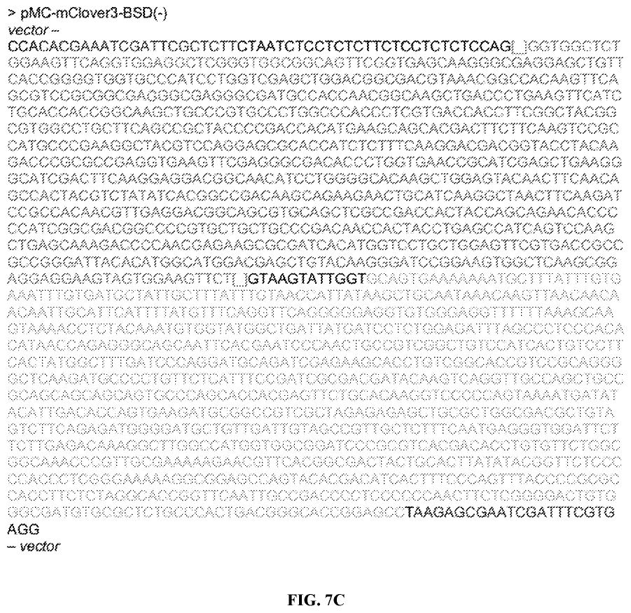

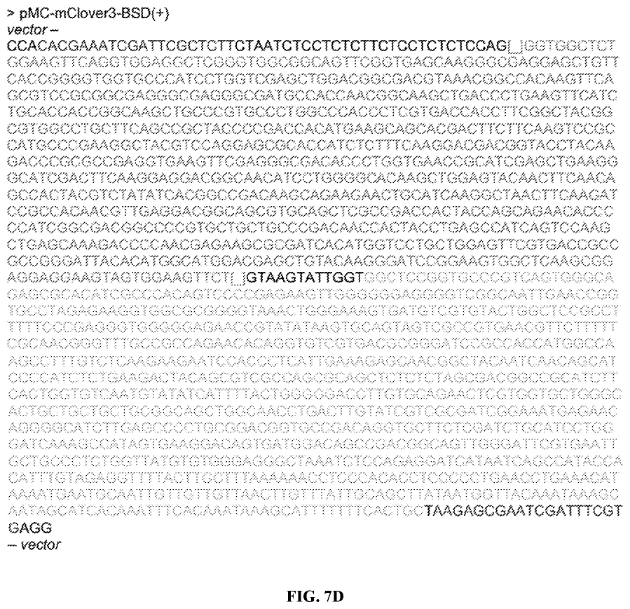

Cloning. The mNG2₁₁ donor tag (Feng et al., 2017) flanked by flexible 15 amino acid linkers was synthesized as two complementary oligos from IDT and annealed. This template was amplified by primers to add splice donor and acceptor sites, sgRNA target sequences external to the splice sites, and 25 nucleotide overhangs into the pMC.BESPX-MCS2 parental vector (System Biosciences). pMC.BESPX-MCS2 was digested with EcoRI and ApaI, and combined with the mNG2₁₁ amplicon by Gibson assembly (NEB), generating the pMC-NG2₁₁ donor plasmid (FIG. 6A; SEQ ID NO: 1). The pMC-mClover3 donor plasmid (FIG. 6B; SEQ ID NO: 2) was generated by replacing the mNG2₁₁ sequence from the pMC-mNG2₁₁ plasmid with the sequence of mClover3 (Addgene #74257) by Gibson assembly. The mNG2₁₁-BSD(−) (FIG. 7A; SEQ ID NO: 3), mNG2₁₁-BSD(+) (FIG. 7B; SEQ ID NO: 4), mClover3-BSD(−) (FIG. 7C; SEQ ID NO: 5), and mClover3-BSD(+) (FIG. 7D; SEQ ID NO: 6) plasmids were generated by inserting DNA encoding the EEF1A1 core promoter, a blasticidin resistance gene, and an SV40 poly(A) sequence in the reverse and forward orientations, respectively, into the pMC-NG2₁₁ or pMC-mClover3 plasmids by Gibson assembly.

To generate HEK293 cells stably expressing mNG2₁₋₁₀, mNG2₁₋₁₀ (Feng et al., 2017) fused to the self-cleaving 2A peptide and tdTomato (Addgene #37347) was cloned into the lenti dCAS-VP64_Blast (Addgene #61425) backbone in place of dCas9-VP64 by 3-piece Gibson assembly.

sgRNA-expressing plasmids (Table 1) were generated by digesting a lentiGuide-Puro plasmid (Addgene #52963) with Esp31 and ligating an annealed sgRNA oligo duplex as described previously (Ran et al., 2013).

TABLE 1
List of sgRNAs used.

| Gene | Intron | Target | Spacer Sequence | SEQ ID NO: |
|---|---|---|---|---|
| VIM | 8 | n/a | CACTAGACTACCTCAATATG | 7 |
| CBX1 | 3 | 1 | TTGGAGTGATTATTCATCAA | 8 |
| CBX1 | 3 | 2 | TTAGTCCTGAAATCTTAGGT | 9 |
| ACTB | 2 | 1 | CCCCCACCCCGGAAACCGGG | 10 |
| ACTB | 2 | 2 | CAAGGGCGCTTTCTCTGCAC | 11 |
| ACTB | 2 | 3 | AGCCTCCCGGTTTCCGGGGT | 12 |
| ACTB | 3 | 1 | GTGGGTGTAGGTACTAACAC | 13 |
| ACTB | 3 | 2 | TAGAACCTGCAGAGTTCCAA | 14 |
| ACTB | 3 | 3 | CCTACTTAATACACACTCCA | 15 |
| ACTB | 5 | 1 | GACAGCTCCCCACACACCAC | 16 |
| ACTB | 5 | 2 | CTGAGCTGACCTGGGCAGGT | 17 |
| ACTB | 5 | 3 | CTGCCCAGGTCAGCTCAGGC | 18 |
| CANX | 10 | 1 | TTGCAACTATAAAAAGACTG | 19 |
| CANX | 10 | 2 | AGATTGTCCAGACTCAGCTG | 20 |
| CANX | 10 | 3 | TTTATAATCTCTACAAAGAG | 21 |
| CANX | 12 | 1 | GGCACAATAAACGGCCACTG | 22 |
| CANX | 12 | 2 | AAAGCTGATTATTGCCCAAG | 23 |
| CANX | 12 | 3 | TAACTAAGATATGTTGCCTG | 24 |
| CANX | 14 | 1 | ATGAACCCATCTATGGACAA | 25 |
| CANX | 14 | 2 | GAGACCAGATTTAGACACAG | 26 |
| CANX | 14 | 3 | ATACTAAAAGTGCTAGAGGT | 27 |
| donor plasmid | n/a | n/a | AAGAGCGAATCGATTTCGTG | 28 |

TABLE 2
List of DNA primers used. ″Orientation″ refers to position of the primer binding
site relative to the sgRNA target site. ″Upstream″ primers were mixed with mNG211
″reverse″ for PCR amplification, and ″downstream″ primers were mixed with mNG211
″forward″.

| | | | | | SEQ |
|---|---|---|---|---|---|
| | | | | | ID |
| Gene | Intron | Target | Orientation | Complementary Sequence | NO: |
| ACTB | 2 | 1 | upstream | CACCAGGTAGGGGAGCTG | 29 |
| ACTB | 2 | 1 | downstream | AGGGTGAGGATGCCTCTCTT | 30 |
| ACTB | 2 | 2 | upstream | CACCAGGTAGGGGAGCTG | 31 |
| ACTB | 2 | 2 | downstream | AGGGTGAGGATGCCTCTCTT | 32 |
| ACTB | 2 | 3 | upstream | CACCAGGTAGGGGAGCTG | 33 |
| ACTB | 2 | 3 | downstream | AGGGTGAGGATGCCTCTCTT | 34 |
| ACTB | 3 | 1 | upstream | TTGCTTTTTCCCAGATGAGC | 35 |
| ACTB | 3 | 1 | downstream | GAACACGGCTAAGTGTGCTG | 36 |
| ACTB | 3 | 2 | upstream | GCCCTTTCTCACTGGTTCTCT | 37 |
| ACTB | 3 | 2 | downstream | GCTTTACACCAGCCTCATGG | 38 |
| ACTB | 3 | 3 | upstream | TTGCTTTTTCCCAGATGAGC | 39 |
| ACTB | 3 | 3 | downstream | GAACACGGCTAAGTGTGCTG | 40 |
| ACTB | 5 | 1 | upstream | CCCAGCACAATGAAGATCAA | 41 |
| ACTB | 5 | 1 | downstream | ACATCTGCTGGAAGGTGGAC | 42 |
| ACTB | 5 | 2 | upstream | GACATCCGCAAAGACCTGTA | 43 |
| ACTB | 5 | 2 | downstream | GTGAGGACCCTGGATGTGAC | 44 |
| ACTB | 5 | 3 | upstream | GACATCCGCAAAGACCTGTA | 45 |
| ACTB | 5 | 3 | downstream | GTGAGGACCCTGGATGTGAC | 46 |
| CANX | 10 | 1 | upstream | TACCCTGCTCTTGGGTGCTA | 47 |
| CANX | 10 | 1 | downstream | AGGCCTAAAGCCTCACAACC | 48 |
| CANX | 10 | 2 | upstream | TACCCTGCTCTTGGGTGCTA | 49 |
| CANX | 10 | 2 | downstream | AGGCCTAAAGCCTCACAACC | 50 |
| CANX | 10 | 3 | upstream | TTAGGCCTCATGCAAAAATG | 51 |
| CANX | 10 | 3 | downstream | GCCAAGATCCTGCTGAAATG | 52 |
| CANX | 12 | 1 | upstream | ACCAAGCCATGTTTGGTGTT | 53 |
| CANX | 12 | 1 | downstream | CAGCAGGCAAAGCTGATTATT | 54 |
| CANX | 12 | 2 | upstream | CCAGATGGGAGCAGGATTTA | 55 |
| CANX | 12 | 2 | downstream | GAAGGTGAAGGCAGAATGGA | 56 |
| CANX | 12 | 3 | upstream | TAGCCCTTCCTGTGTTCCTG | 57 |
| CANX | 12 | 3 | downstream | ACAATAAACGGCCACTGAGG | 58 |
| CANX | 14 | 1 | upstream | TTGCCTCTCCTCACTGTGC | 59 |
| CANX | 14 | 1 | downstream | ACTGCTCATTGCCTGTTTCC | 60 |
| CANX | 14 | 2 | upstream | AGGGTGACAGGAGAGGAACA | 61 |
| CANX | 14 | 2 | downstream | GGAAGGCAGAGTTGTAGCTGA | 62 |
| CANX | 14 | 3 | upstream | GGAAACAGGCAATGAGCAGT | 63 |
| CANX | 14 | 3 | downstream | CACTTACATCCCCATGGAAAA | 64 |
| mNG211 | n/a | n/a | forward | CTCCTCTCTTCTCCTCTCTCCA | 65 |
| mNG211 | n/a | n/a | reverse | AACCAATACTTACAGAACTTCCA | 66 |
| CANX | 14 | 1 | upstream | TGGCACTGTCAGTCAAGAGG | 67 |
| | | | (1921 bp) | | |
| CANX | 14 | 1 | downstream | CGTGGCTTTCTGTTTCTTGG | 68 |
| | | | (1921 bp) | | |

Cell culture and transfections. Transfection experiments were carried out in HEK293 (ATCC CRL-1573), HeLa cells (ATCC CCL-2), H9 hESCs (WiCell), and HAP1 cells (Horizon). The HEK293 cells were generated to constitutively express mNG2₁₋₁₀ and tdTomato from a stably integrated lentiviral cassette. Individual clones were sorted based on the tdTomato signal and a line with stable expression over time was selected for experiments.

HEK293 and HeLa cells were cultured in DMEM (Thermo Fischer Scientific) +10% fetal bovine serum (FBS; VWR) +antibiotic-antimycotic (Thermo Fisher Scientific). HAP1 cells were cultured in IMDM (Thermo Fisher Scientific). H9 cell lines were cultured in a feeder-free system on plates coated with hESC-qualified Matrigel (Corning 354277) and were maintained in mTeSR1 media (STEMCELL Technologies 85850). H9 cells were dissociated using StemPro Accutase (Gibco) and 2×10⁵ cells were replated per well of a 12-well plate in mTeSR1 supplemented with 10 μM ROCK inhibitor (Stemolecule &-27632, Stemgent) for 24 h. Blasticidin selection of HEK293 and HAP1 cells was performed with 5 μg/mL blasticidin (Thermo Fisher Scientific).

For transfection experiments, cells were plated across a 12-well plate such that they would be ˜60% confluent on the day of transfection. The donor plasmid was delivered at 5x the molar ratio of lentiCas9-Blast plasmid (Addgene #53962) and the two lentiGuide-Puro plasmids (Addgene #52963) encoding (1) the donor-cutting sgRNA and (2) the genomic locus-targeting sgRNA (Table 1). In total, ˜1.4 μg of DNA were delivered to each well. For HEK293 and HeLa cells, DNA was delivered in 100 μL, Opti-MEM (Thermo Fischer Scientific) with 4.3 μL, of 1 g/L PEI (Polysciences, cat. #24765). For HAP1 cells, DNA was delivered in 50 μL, Opti-MEM with 3 μL, Lipofectamine Stem reagent (Thermo Fisher Scientific), along with equal amounts relative to the Cas9- and sgRNA-expressing plasmids of the episomal vector expressing TP53 inhibitor (Addgene 41856). After six days, cells were harvested, analyzed, and sorted by flow cytometry.

Flow cytometry and cell sorting. Cultured cells were trypsinized, resuspended in the appropriate media to ˜1×10⁶ cells/mL, and filtered through a cell strainer. Cellular fluorescence was measured on a BD FACSAria Fusion (BD Biosciences). mClover3 and mNG2 fluorescence were detected by the 488 nm laser and filters 502LP and 530/30. Autofluorescence was detected by the 405 nm laser and the 450/50 filter. Polyclonal fluorescent cell populations were acquired by isolating 1000 cells by sorting. Data were analyzed using Flowing Software 2 ver. 2.5.1 (available on the world wide web at flowingsoftware.btk.fi/index.php).

Confocal microscopy and image processing. For imaging experiments, cells were grown on coverslips and directly fixed in 4% formaldehyde (Electron Microscopy Sciences) in PBS (Thermo Fischer Scientific). Fixed cells were washed in PBS and coverslips were mounted on microscopy slides in Vectashield mounting medium (Vector Laboratories). Images were acquired on a Leica TCS SP8 confocal microscope. Z-stacks (0.6 p.m slices) spanning the entire volume of the cells were recorded with oil-immersion 63× x Plan-Apochromat lenses, 1.4 NA. Images were processed using Fiji (Schindelin et al., 2012).

Western blotting. Cultured cells were pelleted, washed with PBS, and resuspended in RIPA lysis buffer (Cell Signaling 9806) with 1× protease inhibitor cocktail (MilliporeSigma P8340). Samples were normalized by bicinchoninic acid (BCA) assay (Cell Signaling 7780), and loaded on a precast SDS-PAGE gel (Bio-Rad 4561086). Western blotting followed using standard protocols. Imaging of blots was performed on a LI-COR Odyssey (LI-COR). The following antibodies were used: α-CANX (Novus Biologicals, NBP2-53352, 1:1000), α-GAPDH (Cell Signaling 2118, 1:2000), IRDye 680LT Goat anti-Rabbit (LI-COR 926-68021, 1:10,000), and IRDye 800CW Goat anti-Mouse (LI-COR 926-32210, 1:10,000).

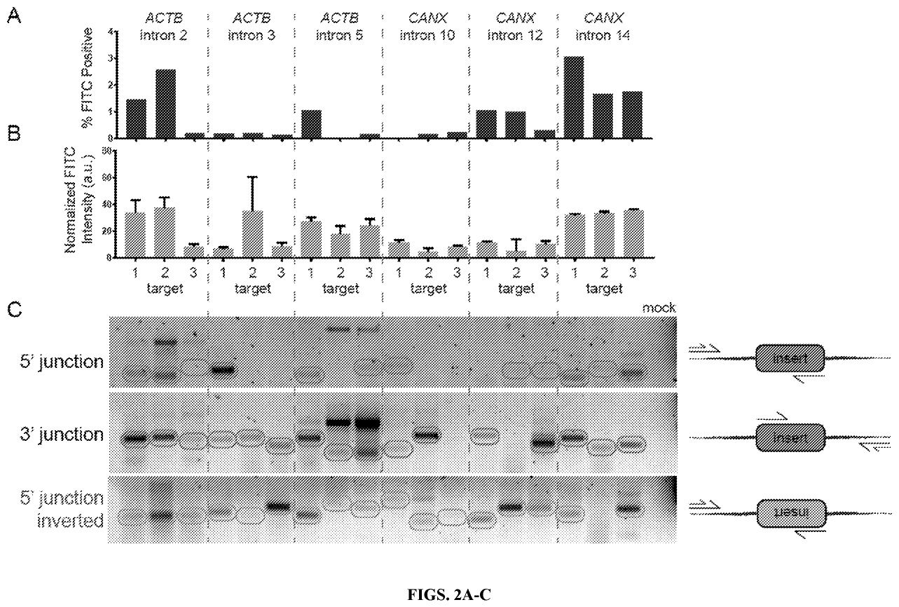

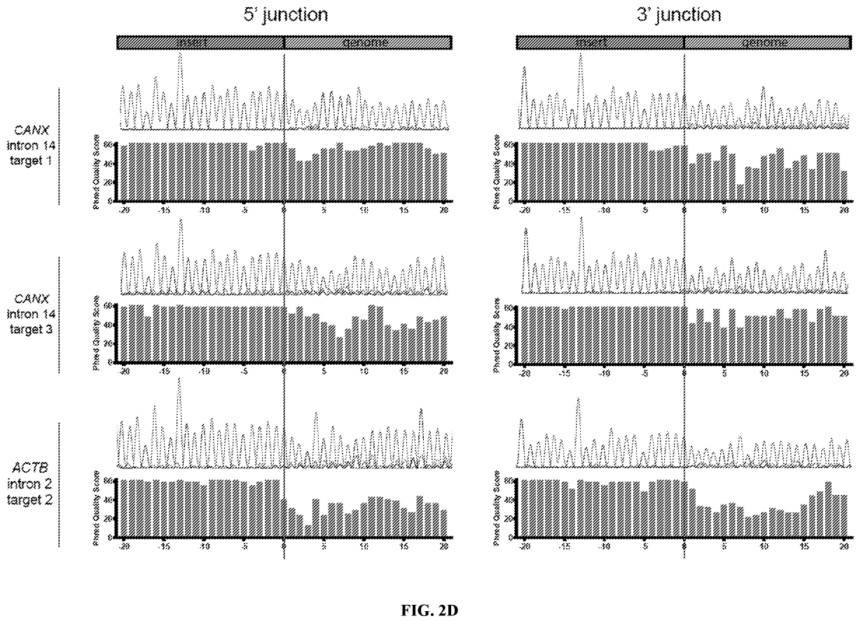

PCR analysis of genomic regions. Roughly 2-3×10⁶ cells were harvested for genomic DNA extraction in 100 μL of QuickExtract (Epicentre) according to the manufacturer's protocol. Amplification of edited genomic regions was performed with the EmeraldAmp MAX PCR Master Mix (Takara Bio USA). For analysis of polyclonal cell populations (FIGS. 2A-D), primers were designed using the default parameters of Primer3 (available at primer3.ut.ee/) to produce amplicons 250-300 nt in length at the 5′ and 3′ junctions of each targeted site. Amplification reactions included a genomic primer upstream of the target integration site paired with a reverse primer hybridizing to the 3′ end of the tag, or a genomic primer downstream from the target integration site with a forward primer hybridizing to the 5′ end of the tag (Table 2). The amplicons were imaged alongside a 100-bp DNA ladder (New England Biolabs) and extracted from a 2% agarose gel using the Monarch Gel Extraction kit (New England Biolabs), and analyzed by Sanger sequencing (GENEWIZ) using the tag-hybridizing primers from the amplification reaction. For analysis of monoclonal cell populations tagged with a longer DNA insert (FIGS. 3E & 5A-C), primers were again designed using Primer3 to produce an amplicon 1921 bp in wild-type cells (Table 2). After amplification, PCR products were run alongside a 1-kb DNA ladder (New England Biolabs).

### Example 2

### Intron-Based Protein Trapping

A plasmid donor was designed that contained the mNG2₁₁ tag, part of a previously-published split fluorophore system (Feng et al., 2017), flanked by linker sequences and splice acceptor (SA) and donor (SD) sites (FIG. 6A; SEQ ID NO: 1). This sequence was embedded between two identical sgRNA target sites, chosen to have minimal off-target activity in the human genome, such that cutting of the plasmid in cells generates a linear DNA donor molecule. Proteins with well-established localization patterns were chosen as targets, and two sgRNAs for two introns for each gene were designed. Plasmids containing SpCas9, sgRNAs against the donor plasmid, the donor plasmid itself, and intron-targeting sgRNAs were transfected into HEK293 cells already stably expressing mNG2₁₋₁₀, which will bind expressed proteins tagged with mNG2₁₁ to emit a fluorescence signal. Multiple introns for each gene were chosen semi-randomly, as the generic nature of the approach allowed for the interrogation of multiple sites at once with minimal additional effort or cost. Intron “frame” was the only criterion that made intron selection non-random: introns that lay precisely in between would-be codons in the adjacent exons were targeted, because the donor used in this study is compatible with frame 1. However, introns that bisect would-be codons would be achieved by using a donor containing the appropriate frameshift mutations.

Using this approach, four tested genes with well-established localization patterns, CANX, CBX1, VIM and ACTB, were tagged at a frequency that enabled easy isolation of both clonal and polyclonal tagged populations of genes (FIG. 1B). To test that this tagging approach was mediated by double-strand breaks in both the genomic sequence and the donor plasmid, each individual component of the transfection mix was removed, and it was found that efficient tagging required all components (FIG. 1C). Then, the feasibility of integrating larger donors was tested by replacing the mNG2₁₁ epitope (˜4.15 kDa) with a full mClover3 fluorescence protein (FP) (˜28.9 kDa) (FIG. 3B), and comparable integration efficiencies were found (FIG. 1D). In the specific case of intron 5 ofACTB, integrating a full-length FP resulted in a lower expression level and a diffuse localization pattern, consistent with the production of non-functional protein (right most panels in FIGS. 1B and 1D). Tagging with a full-length FP versus a split FP is likely to affect the folding dynamics of the targeted protein differently at certain sites, potentially explaining the difference seen with ACTB.

To verify that the observed activity was not specific to HEK293 cells, HeLa cells, H9 human embryonic stem cells, and HAP1 cells were also tagged (FIG. 1E). All of these cell types exhibited tagging efficiencies below 0.5% for either CANX or CBX1 at the conditions tested.

Unsuccessful tagging can be a result of, but not limited to, inefficient genomic DNA cutting, low donor integration, inefficient splicing, or a fusion location that detrimentally affects protein folding. To start investigating these alternatives, two genes, ACTB and CANX, were chosen, and nine sgRNAs were designed for each that spanned three introns. Tagging efficiency and the protein expression levels in pre-enriched, polyclonal tagged cells at each of these locations was measured (FIGS. 2A and 2B). Efficient integration associated with high expression levels of the protein typically coincided within the same intron, indicating that the location of the fusion within the protein is a more critical parameter than the choice of the sgRNA within an intron. Integration of the donor construct appeared to occur for all locations whether or not successful tagging was observed, as analyzed by PCR using genomic templates from the total transfected cell populations and primers that were designed to amplify the genomic DNA-to-donor junction on both sides of the donor (FIG. 2C). Little discernable directional preference was observed for donor integration, and tandem insertions were also observed, as evidenced by the upper bands corresponding to twice and sometimes three times the expected molecular weight of a single insertion (FIG. 2C). Sanger sequencing was used on some of the amplified junctions and accurate integration was found to be sometimes flanked by junction indels (FIG. 2D), further emphasizing the advantages of targeting introns using such an approach.

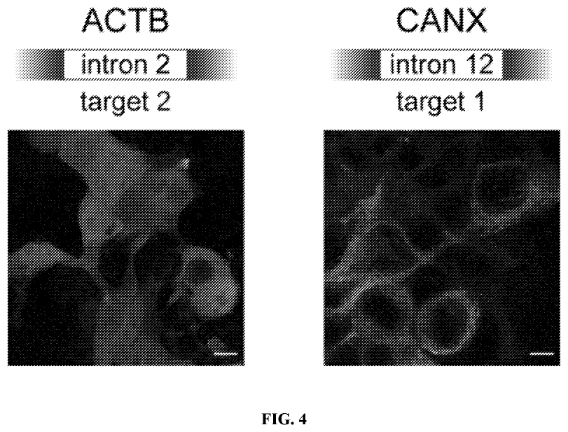

Picking three introns at random for CANX resulted in the identification of two feasible fusion locations that do not disrupt protein localization: at intron 14 (FIG. 1B) and at intron 12 (FIG. 4), emphasizing the ease with which fusion locations can be identified using this approach. However, not all fusions that resulted in high tagging efficiency and fluorescence intensity indicated a successful fusion, as tagging ACTB at intron 2 disrupted proper localization (FIG. 4). Therefore, novel fusion locations should be validated by additional methods.

Additional advantage was taken of the use of splicing for the generation of protein fusions, and a blasticidin resistance gene was added to the donor cassette outside of the splice acceptor and donor sites but still between the donor protospacer sequences such that integration events can be selected without fusing the resistance cassette to the target protein-coding sequence (FIG. 3A). Because the blasticidin resistance gene would be expressed whether or not the donor construct is integrated in the proper orientation, the theoretical maximum percentage of positively tagged cells is 50% after blasticidin selection. This degree of enrichment would be immensely beneficial when tagging efficiency is very low and when isolation of clones without sorting is required (e.g., for nonfluorescent tags). In addition, in cases for which clonal isolation is not possible, increasing the number of tagged cells can facilitate analysis of a polyclonal population.

Because the resistance gene is close to the splice donor and also contains an active promoter, a potential effect on splicing efficiency was anticipated and thus donor cassettes with the resistance gene inverted (mNG2₁₁-BSD(−); FIG. 7A; SEQ ID NO: 3) and in parallel (mNG2₁₁-BSD(+); FIG. 7B; SEQ ID NO: 4) relative to the splice donor site were tested. Tagging of CANX and CBX1 with mNG2₁₁-BSD(−/+) revealed a large increase in the percent of positively tagged cells after 2-3 wk selection with blasticidin (FIG. 3B). CBX1 seemed to benefit more greatly from blasticidin selection than CANX in terms of fold change, potentially owing to locus-specific effects. Although there was no significant difference between mNG2₁₁-BSD(−) and mNG2₁₁-BSD(+) in terms of the percent of positively tagged cells over time, tagging with mNG2₁₁-BSD(−) appeared to result in a fluorescent cell population with an overall higher fluorescence intensity compared to the nonfluorescent population (FIG. 3C). This effect could result from the promoter of the BSD gene interfering more strongly with splicing machinery in the mNG2₁₁-BSD(+) cassette, or attributable to other effects on protein expression. Imaging of cells after blasticidin selection but before sorting confirmed a high efficiency as well as the anticipated protein localization patterns (FIG. 3D), supporting the notion that the BSD gene does not affect the targeted protein function more so than only introducing the fluorescence tag.

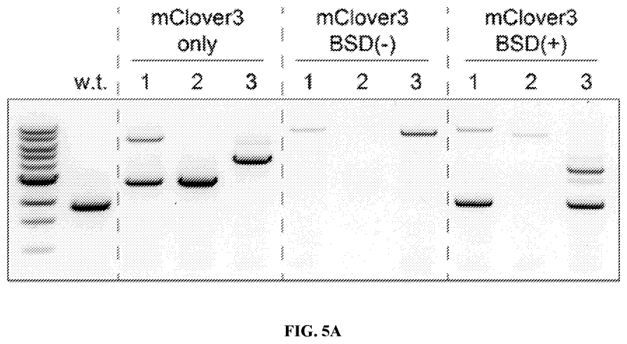

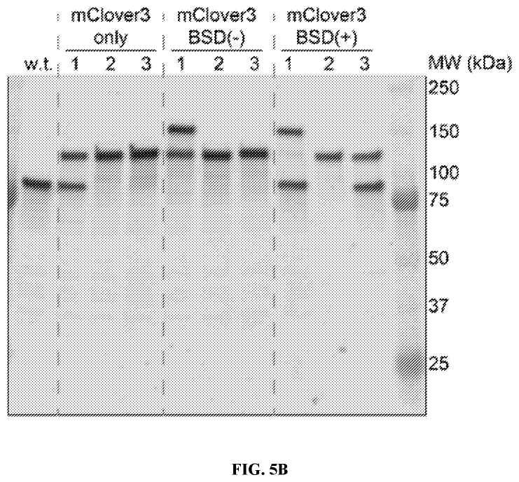

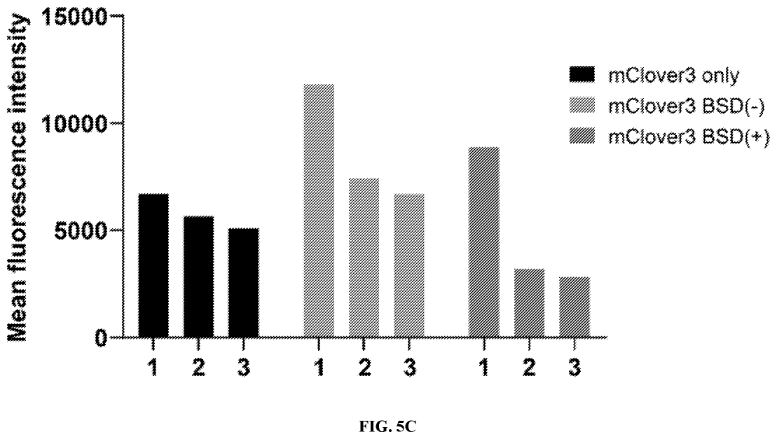

To more thoroughly evaluate donor splicing and protein stability after integration of a large tag and a resistance gene, a BSD-containing mClover3 generic donor (mClover3-BSD(−) (FIG. 7C; SEQ ID NO: 5) and mClover3-BSD(+) (FIG. 7D; SEQ ID NO: 6)) was created. All mClover3-based generic donors were transfected into predominantly haploid populations of HAP1 cells targeting CANX, which after several passages can transform into diploid cells with homozygous tagged alleles (Olbrich et al. 2017). Clonal cell lines were obtained by cell sorting, expanded, and modified CANX protein was analyzed by western blotting. The primary band of most clones corresponded to a single size of CANX, indicating that the splicing efficiency of all donors is virtually 100% (FIG. 3E). Clones with multiple sizes of CANX typically corresponded to cells with heterozygous tag integration as assessed by genomic PCR (FIG. 5A), and none of the tagging led to unexpected protein sizes (FIG. 5B). CANX levels as assessed by the sum of the bands were largely unchanged by tagging, except possibly in the case of mClover3-BSD(+), where protein levels generally appeared lower (FIG. 3E). This was confirmed by flow cytometry analysis of the clones (FIG. 5C) and is consistent with the BSD(+) population appearing dimmer than the BSD(−) population in FIG. 3C. Taken together with the imaging data, internally tagging endogenous genes by intron-targeted protein trapping can be performed without necessarily disrupting protein localization or stability, even in the presence of a proximal resistance gene.

Generating endogenous fusions by HITI-mediated intron tagging is efficient and easy to implement. As editing is done in introns, this approach tolerates indels both in the untagged allele and in the sequences that flank the donor integration site. A small number of sgRNAs, spanning multiple introns, is sufficient to identify a successful tagging site, and as these do not require a loci-specific donor, costs are minimal. This approach simplifies the generation of knock-in cell lines and makes scalable gene tagging highly accessible.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bürckstümmer et al., A reversible gene trap collection empowers haploid genetics in human cells. Nat Methods 10: 965-971 (2013).
Buszczak et al., The carnegie protein trap library: a versatile tool for Drosophila developmental studies. Genetics 175: 1505-1531 (2007).
Cabantous et al., Protein tagging and detection with engineered self-assembling fragments of green fluorescent protein. Nat Biotechnol 23: 102-107 (2005).
Clyne et al., Green fluorescent protein tagging Drosophila proteins at their native genomic loci with small P elements. Genetics 165, 1433-1441 (2003).
Cohen et al., Dynamic proteomics of individual cancer cells in response to a drug. Science 322: 1511-1516 (2008).
Doudna & Charpentier, Genome editing. The new frontier of genome engineering with CRISPR-Cas9. Science 346: 1258096 (2014).
Feng et al., Improved split fluorescent proteins for endogenous protein labeling. Nat Commun 8: 370 (2017).
Hsu et al., Development and applications of CRISPR-Cas9 for genome engineering. Cell 157: 1262-1278 (2014).
Jarvik et al., CD-tagging: a new approach to gene and protein discovery and analysis. Biotechniques 20: 896-904 (1996).
Kamiyama et al., Versatile protein tagging in cells with split fluorescent protein. Nat Commun 7: 11046 (2016).
Lackner et al., A generic strategy for CRISPR-Cas9-mediated gene tagging. Nat Commun 6: 10237 (2015).
Leonetti et al., A scalable strategy for high-throughput GFP tagging of endogenous human proteins. Proc Natl Acad Sci USA 113: E3501-3508 (2016).
Olbrich et al., A p53-dependent response limits the viability of mammalian haploid cells. Proc Natl Acad Sci 114: 9367-9372 (2017).
Ran et al., Genome engineering using the CRISPR-Cas9 system. Nat Protoc 8: 2281-2308 2013).
Schindelin et al., Fiji: an open-source platform for biological-image analysis. Nat Methods 9: 676-682 (2012).
Schmid-Burgk et al., CRISPaint allows modular base-specific gene tagging using a ligase-4-dependent mechanism. Nat Commun 7: 12338 (2016).
Sigal et al., Dynamic proteomics in individual human cells uncovers widespread cell-cycle dependence of nuclear proteins. Nat Methods 3: 525-531 (2006).
Sigal et al., Generation of a fluorescently labeled endogenous protein library in living human cells. Nat Protoc 2: 1515-1527 (2007).
Suzuki et al., In vivo genome editing via CRISPR/Cas9 mediated homology-independent targeted integration. Nature 540: 144-149 (2016).
Trinh le & Fraser, Enhancer and gene traps for molecular imaging and genetic analysis in zebrafish. Dev Growth Differ 55: 434-445 (2013).
Trinh le et al., A versatile gene trap to visualize and interrogate the function of the vertebrate proteome. Genes Dev 25: 2306-2320 (2011).

1. A nucleic acid comprising, from 5′ to 3′, a first sgRNA binding site, a splice acceptor site, a sequence encoding a reporter protein, a splice donor site, and a second sgRNA binding site.

2. The nucleic acid of claim 1, wherein the first and second sgRNA binding sites comprise the same nucleotide sequence.

3. The nucleic acid of claim 1, wherein the reporter protein is a fluorescent protein.

4. The nucleic acid of claim 3, wherein the fluorescent protein is a split fluorescent protein fragment.

5. The nucleic acid of claim 1, further comprising an antibiotic resistance gene positioned between the splice donor site and the second sgRNA binding site or between the first sgRNA binding site and the splice acceptor site.

6. The nucleic acid of claim 5, wherein the antibiotic resistance gene is blasticidin.

7. A composition comprising an endonuclease-encoding nucleic acid sequence, the donor plasmid of claim 1, and a donor plasmid-specific gRNA-encoding sequence.

8. The composition of claim 7, wherein the endonuclease is a Cas endonuclease.

9. The composition of claim 8, wherein the Cas endonuclease is a Cas9 endonuclease.

10. The composition of claim 7, further comprising a site-specific guide RNA (gRNA)-encoding nucleic acid sequence.

11. The composition of claim 7, wherein the site-specific and/or donor plasmid-specific guide RNA is a single gRNA.

12. The composition of claim 11, wherein the site-specific and/or donor plasmid-specific guide RNA is a CRISPR-RNA (crRNA).

13. The composition of claim 12, wherein the site-specific and/or donor plasmid-specific guide RNA comprises a fusion of a crRNA and a trans-activating CRISPR RNA (tracrRNA).

14. The composition of claim 7, wherein the guide RNA comprises a crRNA and a tracrRNA.

15. The composition of claim 7, wherein the endonuclease and the donor plasmid-specific gRNA are encoded on a single nucleic acid molecule.

16. The composition of claim 7, wherein the endonuclease, the donor plasmid-specific gRNA, and the donor plasmid are encoded on a single nucleic acid molecule.

17. The composition of claim 7, wherein each of the endonuclease-encoding nucleic acid sequence, the donor plasmid, the site-specific guide RNA (gRNA)-encoding nucleic acid sequence, and the donor plasmid-specific gRNA-encoding sequence are present on separate nucleic acid molecules.

18. A method of integrating an exogenous DNA sequence into an intronic genomic sequence of a target gene in a cell, the method comprising delivering a composition comprising delivering to the cell a composition of claim 7.

19. The method of claim 18, wherein the portion of the donor plasmid comprising the splice acceptor site, the sequence encoding a reporter protein, and the splice donor site is integrated into the intronic genomic sequence of a target gene.

20. The method of claim 19, wherein the reporter protein is expressed when the target gene is expressed.

21. The method of claim 18, wherein the portion of the donor plasmid comprising the splice acceptor site, the sequence encoding a reporter protein, the splice donor site, and the antibiotic resistance gene is integrated into the intronic genomic sequence of a target gene.

22. The method of claim 19, further comprising detecting the expression of the antibiotic resistance gene.

23. The method of claim 18, further comprising detecting the expression of the reporter protein.

24. The method of claim 18, wherein the method comprises integrating the exogenous DNA sequence into an intronic genomic sequence of a second target gene in a second cell.

25. The method of claim 24, wherein the method is further defined as a high-throughput method of tagging target genes, wherein the method comprises integrating the exogenous DNA sequence into an intronic genomic sequence in two or more cells, wherein the intronic genomic sequence is unique for each of the two or more cells.


SEQUENCE LISTING (68 sequences)

SEQ ID NO: 1 - DNA, 225 bp, Artificial Sequence
  Synthetic polynucleotide
ccacacgaaatcgattcgctcttctaatctcctctcttctcctctctccagggtggctctggaagttcaggtggaggctcgggtggcggcagttcgaccgagctcaacttcaaggagtggcaaaaggcctttaccgatatgatgggatccggaagtggctcaagcggaggaggaagtagtggaagttctgtaagtattggttaagagcgaatcgatttcgtgagg

SEQ ID NO: 2 - DNA, 891 bp, Artificial Sequence
  Synthetic polynucleotide
ccacacgaaatcgattcgctcttctaatctcctctcttctcctctctccagggtggctctggaagttcaggtggaggctcgggtggcggcagttcggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagggatccggaagtggctcaagcggaggaggaagtagtggaagttctgtaagtattggttaagagcgaatcgatttcgtgagg

SEQ ID NO: 3 - DNA, 1078 bp, Artificial Sequence
  Synthetic polynucleotide
ccacacgaaatcgattcgctcttctaatctcctctcttctcctctctccagggtggctctggaagttcaggtggaggctcgggtggcggcagttcgaccgagctcaacttcaaggagtggcaaaaggcctttaccgatatgatgggatccggaagtggctcaagcggaggaggaagtagtggaagttctgtaagtattggtgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagatttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctaagagcgaatcgatttcgtgagg

SEQ ID NO: 4 - DNA, 1078 bp, Artificial Sequence
  Synthetic polynucleotide
ccacacgaaatcgattcgctcttctaatctcctctcttctcctctctccagggtggctctggaagttcaggtggaggctcgggtggcggcagttcgaccgagctcaacttcaaggagtggcaaaaggcctttaccgatatgatgggatccggaagtggctcaagcggaggaggaagtagtggaagttctgtaagtattggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaatctccagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgctaagagcgaatcgatttcgtgagg

SEQ ID NO: 5 - DNA, 1744 bp, Artificial Sequence
  Synthetic polynucleotide
ccacacgaaatcgattcgctcttctaatctcctctcttctcctctctccagggtggctctggaagttcaggtggaggctcgggtggcggcagttcggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagggatccggaagtggctcaagcggaggaggaagtagtggaagttctgtaagtattggtgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagatttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctaagagcgaatcgatttcgtgagg

SEQ ID NO: 6 - DNA, 1744 bp, Artificial Sequence
  Synthetic polynucleotide
ccacacgaaatcgattcgctcttctaatctcctctcttctcctctctccagggtggctctggaagttcaggtggaggctcgggtggcggcagttcggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagggatccggaagtggctcaagcggaggaggaagtagtggaagttctgtaagtattggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaatctccagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgctaagagcgaatcgatttcgtgagg

SEQ ID NO: 7 - DNA, 20 bp, Artificial Sequence
  Synthetic polynucleotide
cactagactacctcaatatg

SEQ ID NO: 8 - DNA, 20 bp, Artificial Sequence
  Synthetic polynucleotide
ttggagtgattattcatcaa

SEQ ID NO: 9 - DNA, 20 bp, Artificial Sequence
  Synthetic polynucleotide
ttagtcctgaaatcttaggt

SEQ ID NO: 10 - DNA, 20 bp, Artificial Sequence
  Synthetic polynucleotide
cccccaccccggaaaccggg

SEQ ID NO: 11 - DNA, 20 bp, Artificial Sequence
  Synthetic polynucleotide
caagggcgctttctctgcac

SEQ ID NO: 12 - DNA, 20 bp, Artificial Sequence
  Synthetic polynucleotide
agcctcccggtttccggggt

SEQ ID NO: 13 - DNA, 20 bp, Artificial Sequence
  Synthetic polynucleotide
gtgggtgtaggtactaacac

SEQ ID NO: 14 - DNA, 20 bp, Artificial Sequence
  Synthetic polynucleotide
tagaacctgcagagttccaa

SEQ ID NO: 15 - DNA, 20 bp, Artificial Sequence
  Synthetic polynucleotide
cctacttaatacacactcca

SEQ ID NO: 16 - DNA, 20 bp, Artificial Sequence
  Synthetic polynucleotide
gacagctccccacacaccac

SEQ ID NO: 17 - DNA, 20 bp, Artificial Sequence
  Synthetic polynucleotide
ctgagctgacctgggcaggt

SEQ ID NO: 18 - DNA, 20 bp, Artificial Sequence
  Synthetic polynucleotide
ctgcccaggtcagctcaggc

SEQ ID NO: 19 - DNA, 20 bp, Artificial Sequence
  Synthetic polynucleotide
ttgcaactataaaaagactg

SEQ ID NO: 20 - DNA, 20 bp, Artificial Sequence
  Synthetic polynucleotide
agattgtccagactcagctg

SEQ ID NO: 21 - DNA, 20 bp, Artificial Sequence
  Synthetic polynucleotide
tttataatctctacaaagag

SEQ ID NO: 22 - DNA, 20 bp, Artificial Sequence
  Synthetic polynucleotide
ggcacaataaacggccactg

SEQ ID NO: 23 - DNA, 20 bp, Artificial Sequence
  Synthetic polynucleotide
aaagctgattattgcccaag

SEQ ID NO: 24 - DNA, 20 bp, Artificial Sequence
  Synthetic polynucleotide
taactaagatatgttgcctg

SEQ ID NO: 25 - DNA, 20 bp, Artificial Sequence
  Synthetic polynucleotide
atgaacccatctatggacaa

SEQ ID NO: 26 - DNA, 20 bp, Artificial Sequence
  Synthetic polynucleotide
gagaccagatttagacacag

SEQ ID NO: 27 - DNA, 20 bp, Artificial Sequence
  Synthetic polynucleotide
atactaaaagtgctagaggt

SEQ ID NO: 28 - DNA, 20 bp, Artificial Sequence
  Synthetic polynucleotide
aagagcgaatcgatttcgtg

SEQ ID NO: 29 - DNA, 18 bp, Artificial Sequence
  Synthetic polynucleotide
caccaggtaggggagctg

SEQ ID NO: 30 - DNA, 20 bp, Artificial Sequence
  Synthetic polynucleotide
agggtgaggatgcctctctt

SEQ ID NO: 31 - DNA, 18 bp, Artificial Sequence
  Synthetic polynucleotide
caccaggtaggggagctg

SEQ ID NO: 32 - DNA, 20 bp, Artificial Sequence
  Synthetic polynucleotide
agggtgaggatgcctctctt

SEQ ID NO: 33 - DNA, 18 bp, Artificial Sequence
  Synthetic polynucleotide
caccaggtaggggagctg

SEQ ID NO: 34 - DNA, 20 bp, Artificial Sequence
  Synthetic polynucleotide
agggtgaggatgcctctctt

SEQ ID NO: 35 - DNA, 20 bp, Artificial Sequence
  Synthetic polynucleotide
ttgctttttcccagatgagc

SEQ ID NO: 36 - DNA, 20 bp, Artificial Sequence
  Synthetic polynucleotide
gaacacggctaagtgtgctg

SEQ ID NO: 37 - DNA, 21 bp, Artificial Sequence
  Synthetic polynucleotide
gccctttctcactggttctct

SEQ ID NO: 38 - DNA, 20 bp, Artificial Sequence
  Synthetic polynucleotide
gctttacaccagcctcatgg

SEQ ID NO: 39 - DNA, 20 bp, Artificial Sequence
  Synthetic polynucleotide
ttgctttttcccagatgagc

SEQ ID NO: 40 - DNA, 20 bp, Artificial Sequence
  Synthetic polynucleotide
gaacacggctaagtgtgctg

SEQ ID NO: 41 - DNA, 20 bp, Artificial Sequence
  Synthetic polynucleotide
cccagcacaatgaagatcaa

SEQ ID NO: 42 - DNA, 20 bp, Artificial Sequence
  Synthetic polynucleotide
acatctgctggaaggtggac

SEQ ID NO: 43 - DNA, 20 bp, Artificial Sequence
  Synthetic polynucleotide
gacatccgcaaagacctgta

SEQ ID NO: 44 - DNA, 20 bp, Artificial Sequence
  Synthetic polynucleotide
gtgaggaccctggatgtgac

SEQ ID NO: 45 - DNA, 20 bp, Artificial Sequence
  Synthetic polynucleotide
gacatccgcaaagacctgta

SEQ ID NO: 46 - DNA, 20 bp, Artificial Sequence
  Synthetic polynucleotide
gtgaggaccctggatgtgac

SEQ ID NO: 47 - DNA, 20 bp, Artificial Sequence
  Synthetic polynucleotide
taccctgctcttgggtgcta

SEQ ID NO: 48 - DNA, 20 bp, Artificial Sequence
  Synthetic polynucleotide
aggcctaaagcctcacaacc

SEQ ID NO: 49 - DNA, 20 bp, Artificial Sequence
  Synthetic polynucleotide
taccctgctcttgggtgcta

SEQ ID NO: 50 - DNA, 20 bp, Artificial Sequence
  Synthetic polynucleotide
aggcctaaagcctcacaacc

SEQ ID NO: 51 - DNA, 20 bp, Artificial Sequence
  Synthetic polynucleotide
ttaggcctcatgcaaaaatg

SEQ ID NO: 52 - DNA, 20 bp, Artificial Sequence
  Synthetic polynucleotide
gccaagatcctgctgaaatg

SEQ ID NO: 53 - DNA, 20 bp, Artificial Sequence
  Synthetic polynucleotide
accaagccatgtttggtgtt

SEQ ID NO: 54 - DNA, 21 bp, Artificial Sequence
  Synthetic polynucleotide
cagcaggcaaagctgattatt

SEQ ID NO: 55 - DNA, 20 bp, Artificial Sequence
  Synthetic polynucleotide
ccagatgggagcaggattta

SEQ ID NO: 56 - DNA, 20 bp, Artificial Sequence
  Synthetic polynucleotide
gaaggtgaaggcagaatgga

SEQ ID NO: 57 - DNA, 20 bp, Artificial Sequence
  Synthetic polynucleotide
tagcccttcctgtgttcctg

SEQ ID NO: 58 - DNA, 20 bp, Artificial Sequence
  Synthetic polynucleotide
acaataaacggccactgagg

SEQ ID NO: 59 - DNA, 19 bp, Artificial Sequence
  Synthetic polynucleotide
ttgcctctcctcactgtgc

SEQ ID NO: 60 - DNA, 20 bp, Artificial Sequence
  Synthetic polynucleotide
actgctcattgcctgtttcc

SEQ ID NO: 61 - DNA, 20 bp, Artificial Sequence
  Synthetic polynucleotide
agggtgacaggagaggaaca

SEQ ID NO: 62 - DNA, 21 bp, Artificial Sequence
  Synthetic polynucleotide
ggaaggcagagttgtagctga

SEQ ID NO: 63 - DNA, 20 bp, Artificial Sequence
  Synthetic polynucleotide
ggaaacaggcaatgagcagt

SEQ ID NO: 64 - DNA, 21 bp, Artificial Sequence
  Synthetic polynucleotide
cacttacatccccatggaaaa

SEQ ID NO: 65 - DNA, 22 bp, Artificial Sequence
  Synthetic polynucleotide
ctcctctcttctcctctctcca

SEQ ID NO: 66 - DNA, 23 bp, Artificial Sequence
  Synthetic polynucleotide
aaccaatacttacagaacttcca

SEQ ID NO: 67 - DNA, 20 bp, Artificial Sequence
  Synthetic polynucleotide
tggcactgtcagtcaagagg

SEQ ID NO: 68 - DNA, 20 bp, Artificial Sequence
  Synthetic polynucleotide
cgtggctttctgtttcttgg